(19) United States
(12) Patent Application Publication
(10) Pub. No.: US 2018/0193580 A1
(43) Pub. Date: Jul. 12, 2018

(54) Baloonless Endotracheal Tube

(71) Applicant: Mehmet Fatih DEMÌRCÌ, Augusta, GA (US)

(72) Inventor: Mehmet Fatih DEMÌRCÌ, Augusta, GA (US)

(21) Appl. No.: 15/128,883

(22) PCT Filed: Mar. 24, 2015
(86) PCT No.: PCT/TR2015/000127

§ 371 Date: Sep. 23, 2016

(30) Foreign Application Priority Data
Mar. 25, 2014 (TR) 2014/03423

(51) Int. Cl. A61M 16/04

(52) U.S. Cl. CPC A61M 16/0447; A61M 16/0402; A61M 16/0479; A61M 16/0459

(57) ABSTRACT

Endotracheal tubes with balloons (cuffed) deform the circulation of air by applying pressure on the walls of the trachea and cause numerous complications. In the endotracheal tube analyzed in the invention, the valve system in the form of flexible membrane is used instead of balloons, during each expiration, the pressure on the trachea wall is decreased and the disorder of getting blood occurring in the mucosa of the trachea was prevented. Moreover, the leaking of the liquid contents coming from in-mouth and stomach into the lungs and the formation of bacteria and the risk of infection was decreased through ventilating the trachea in a more physiological manner.

This invention is the balloon-less endotracheal tube, which decreases risk of infections, aspiration and tracheal ischemia.

### BACKGROUND OF THE INVENTION

In order to provide respiratory support for patients with advanced respiratory insufficiency, an endotracheal tube is inserted into the trachea (windpipe) and the patient is connected a mechanical ventilator. One edge of the endotracheal tube reaches out into the trachea while the other edge is connected to the ventilator attachment unit.

In the clinical setting, the endotracheal tubes used for adult patients consist of a balloon (cuff), which is located in the tube body at the end of the trachea. The balloon prevents the leaking of air by blocking the gap between trachea and the tube. The balloon is then inflated through a separate cannula, which opens into the edge of the tube in the external environment and a valve. This balloon is important during clinical scenarios where patients require high levels of air pressure in the lungs. In addition to significant functions, the endotracheal tube balloon has side effects as well.

During clinical scenarios where the balloon is inflated over a long period of time, it may lead to blood staining in the mucosa and walls of the trachea, infections, and tracheal injuries, such as tracheal stenosis and fistulas. Moreover; the dead space between the tube and the trachea also causes bacterial proliferation thus more infections.

### REFERENCES

Barash, P G; Cullen, B F; Stoelting, R K, eds. (2009). Clinical Anesthesia (6th ed.). Philadelphia: Lippincott Williams & Wilkins. ISBN 978-0-7817-8763-5.

Benumof, J L, ed. (2007). Benumofs Airway Management: Principles and Practice (2nd ed.). Philadelphia: Mosby-Elsevier. ISBN 978-0-323-02233-0.

Sengupta, P; Sessler, D I; Maglinger, P; Wells, S; Vogt, A; Durrani, J; Wadhwa, A (2004). “Endotracheal tube cuff pressure in three hospitals, and the volume required to produce an appropriate cuff pressure” (PDF). BMC Anesthesiology. 4 (1): 8. doi:10.1186/1471-2253-4-8. PMC 535565. PMID 15569386.

### BRIEF SUMMARY OF THE INVENTION

The objective of this invention is to establish an endotracheal tube that can block air without using a balloon. This will help to prevent ischemic deformation in the trachea caused by endotracheal tubes while the balloon is in use.

Another objective of the invention is to decrease the risk of infection by providing continuous air flow between the tube and trachea, establishing a more physiological environment, and decreasing the bacteria growth. The device will also serve to absorb and remove fluids, such as gastric contents, that accumulate between the ET tube and trachea, providing another method of decreasing the risk of infection.

Finally the innovation may decrease the risk of infection through absorbing the fluids which have accumulated in the dead space between the tube and trachea which will remove it from the environment.

The intention of this invention is also to decrease the risk of infection in the lungs through preventing the passage of fluids which is accumulated between the tube and the trachea which comes from the stomach up to the mouth into the lungs.

### BRIEF DESCRIPTION OF DRAWINGS

FIG. 1: The outlook of the endotracheal tube without a balloon related to the invention.

FIG. 2: The outlook of the flexible membranes which function as valves are connected to the main body of the tube, the balloons which expand the membranes, cleaning openings and balloon tubes.

FIG. 3: The outlook of the tube inside the trachea when the flexible membranes are opened.

FIG. 4: The outlook of the endotracheal tube when the edge towards the patient and the valve membranes are closed.

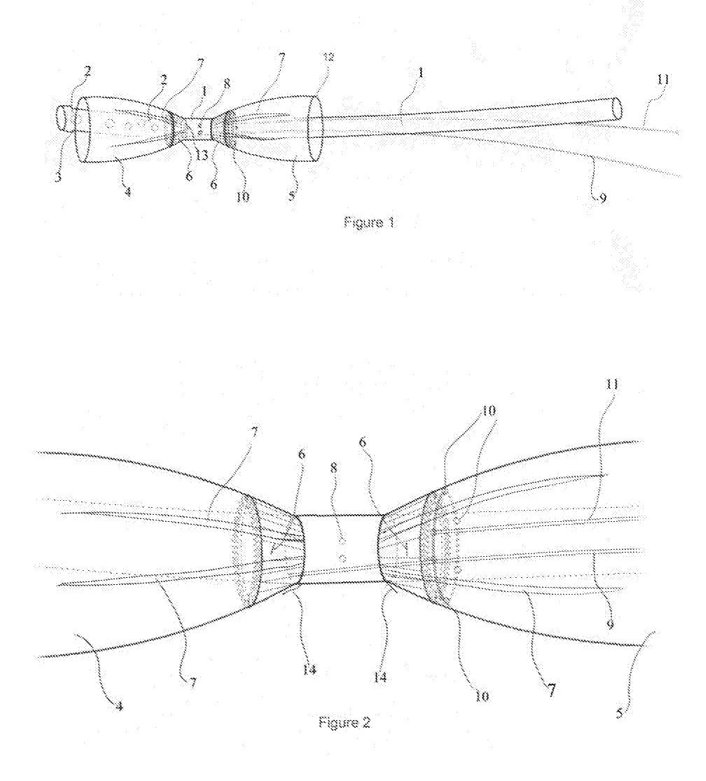

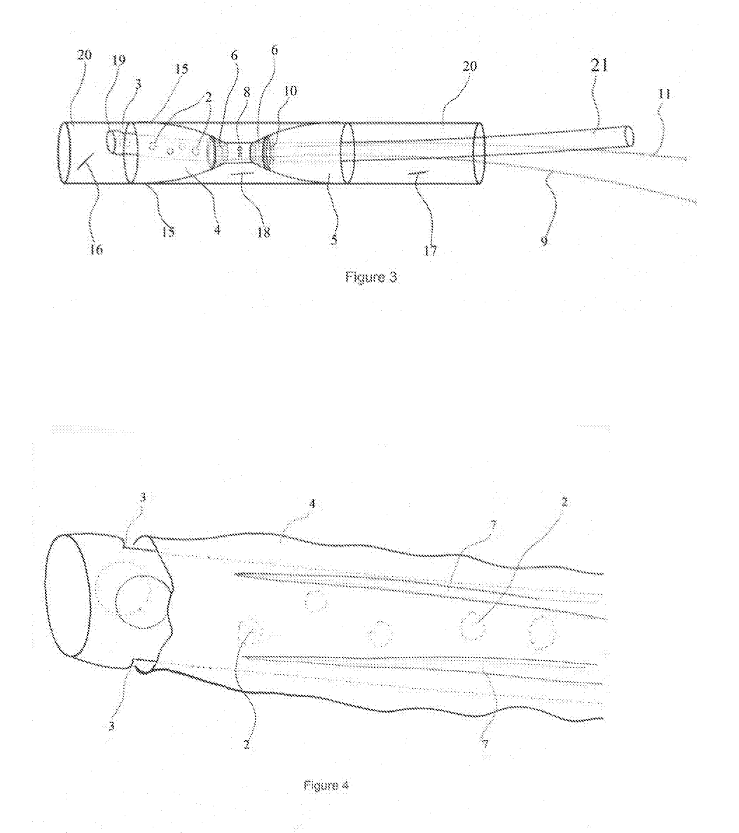

### THE EXPLANATION OF THE REFERENCES IN THE DRAWINGS

1: The main body of the tube
2: The vent-hole at the edge of the tube on the side towards the patient
3: The indentation of the flexible membrane on the edge towards the patient
4: The flexible membrane valve towards the lungs
5: The flexible membrane valve towards the pharynx
6: The balloons which open the valve of the flexible membranes
7: The framework bar of the flexible membrane valves
8: The ventilation holes of the tube body
9: The inflation tube for valve balloons
10: The cleanout holes towards the pharynx
11: The cleanout tube towards the pharynx
12: The bottom of the flexible membrane valve cone
13: The top of the flexible membrane valve cone
14: The junction angle between the flexible membrane valve and the tube body
15: The contact zone of trachea wall with the flexible membrane valve
16: The gap which is limited by the flexible membrane valve between the lungs and towards the lungs
17: The gap, which opens to the pharynx, is limited by the flexible membrane valve towards the pharynx
18: The dead space within the trachea between two valves
19: The endotracheal tube which goes towards the patient
20: Trachea
21: The endotracheal tube towards the machine

### DETAILED DESCRIPTION OF THE INVENTION

The endotracheal tube basically consists of;

The main body of the tube (1)

The holes on the tube towards the patient (2)

The wall indentation on the tube towards the patient so that it comprises the flexible membrane valve when it is closed. (3)

The pulmonary valve membrane of the tube at the edge towards the patient (4)

The pharynx valve membrane at the edge of the tube towards the machine (5)

The balloons which is located on the joint of two valve membranes also the tube which opens the valve membranes (6)

The framework rods which protects the form of the valve membranes (7)

The ventilation holes on the tube body between the two valve membranes (8)

The inflation tube for inflating the valve membranes on the tube towards the machine (9)

The cleanout holes between the endotracheal tube and trachea towards the pharynx which opens the junction angle of the valve membrane also the tube body towards the pharynx (10)

The cleanout tube for the space between the endotracheal tube and the trachea which is towards the pharynx; is open on the edge of the tube towards the machine (11).

The main body of the tube (1) has the similar size and material characteristics as the endotracheal tube. The pulmonary valve membrane of the tube towards the patient (4) basically consists of a curved and conic structure which covers the outside of the endotracheal tube. The bottom of the cone towards the lungs (12) is free while the top of it (13) is attached to the tube so that it covers the outside of the endotracheal tube. The free bottom of the cone has the form which turns into a cylindrical shape towards the bottom. The valve membrane has a flexible body which is made of materials strong enough to keep its original structure. There are balloons (16) which cover the tube like a ring that are located at the junction angles of valve membranes and the endotracheal tube (14). The 6-8 piece rods (7) which lie between the balloon and the bottom of the membrane cone and which allow the framework structure to be harder than the material of the valve membrane are adjacent to the valve. The rods were designed to be tapered towards the bottom in order to protect the convenient curve of the valve membrane. Prior to the installation of the tube into the trachea, the balloons (6) which open the valve membranes (4,5) are completely flat. In this location, the valve membrane completely covers the endotracheal tube and locates precisely (4) behind the indentation (3) on the tube towards the patient (FIG. 4). After the endotracheal tube is inserted into trachea, the balloons which open the valve membrane through the inflation tube (9) for the valve membrane balloons. The conic valve membranes (4,5) which take the cylindrical shape towards the bottom are in contact with the tracheal wall throughout their free edges when the conic valve membranes (4,5) are in the open status (3).

When the air is sent to the lungs through endotracheal tube (in the inspiration manner); the pressure within the space (16) limited by the lungs and the valve membrane towards the lungs increases. The increasing air pressure in the lungs pushes the valve membrane towards the trachea walls (15). The two planes consisting of trachea walls and valve membrane are parallel and adjacent to each other. Therefore, the air in the lungs is prevented from leaking out. When the air in the lungs is expired through the endotracheal tube (expiration phase), the air pressure in the space (16) limited with the lungs and the valve membrane towards the lungs decreases. Since the air pressure which pushes the valve membrane towards the trachea walls decreases, the blood circulation in the trachea walls and mucosa turns to normal. This cycle continues at each phase of the inspiration and expiration, the blood circulation of the trachea mucosa continues. Due to this, the tracheal deformations and complications which were caused by the balloons of the traditional endotracheal tubes are prevented.

The valve membrane (5) towards the pharynx has the same structure and aspects as the valve membrane towards the lungs and there are also cleanout holes (10) which open to the angle between the membrane and the tube body.

On the occasions when the patient is able to breathe spontaneously the mechanism functions differently. In the inspiration phase the air pressure in this space which is limited by the lungs and the valve membrane towards the lungs decreases to negative values. The valve membrane towards the lungs becomes dysfunctional, when the valve membrane towards the pharynx (5) reverse it will then become functional. The external air pressure which is higher than the pressure in the lungs pushes the pharynx side valve membrane towards the trachea walls. Two surfaces of the trachea wall and valve membrane towards the pharynx are adjacently located in a large area parallel to each other. The air enters into the lungs through the endotracheal tube; this has a much lower resistance compared to the space between the tube and trachea.

In the phase of expiration, the high air pressure in the space which is limited by lungs and the valve membrane towards the lung, keeps the valve membrane functioning. Thus the air can be evacuated through the endotracheal tube.

In summary, there is positive pressure which increases during the inspiration phase and decreases during the expiration phase within the space between the lungs and the valve membrane towards lungs, when breathing is provided by the mechanical ventilator and related devices. In both phases, the valve membranes towards the lungs are activated and prevent the air leaking through the gap between the endotracheal tube and the trachea. During this function, as the pressure of the valve membrane to the tracheal mucosa discontinues, the mucosa and the other structures of the trachea can get sufficient blood flow. By this means, the possible complications related to tracheal wall ischemia are prevented. On the occasions where the patient can breathe without respiratory support (spontaneous breathing); the valve membrane towards the pharynx functions and blocks the air leak through the gap between the endotracheal tube and the trachea while inhaling (inspiration phase) and the other valve membrane does not apply pressure to the trachea wall. While in the (expiration phase), the valve membrane towards the lungs functions while the other valve membrane is not apply pressure to the trachea. In this manner, the mucosa and the other structures of the trachea are allowed to get better blood circulation, as there is no constant pressure on the tracheal wall. By this means, possible complications are prevented.

Another function of the valve membrane towards the pharynx is to prevent liquids from inside mouth or stomach leaking to the lungs. An additional benefit of this is the possibility to clean these liquids up. The contents of the stomach may easily leak into the trachea when it reaches up to pharynx through esophagus. Similarly, the liquids accumulating in the mouth may also leak into the trachea. Those items which may cause very dangerous infections when they enter the lungs through the trachea are collected by the valve membrane towards the pharynx. The liquids collected through the holes (10) at the angle where the membrane is connected to the tube body and the slim tube (11) which those holes are connected to. Even, the gap (17) between the trachea opening to the pharynx and endotracheal tube may be washed by water to be given through the same tube (11) and the holes (10). The water is removed from the environment through in-mouth and re-washing tube (11) and holes (10). Thus the formation bacterial proliferation is prevented and the risk of infections are decreased.

The small diameter holes (8) between two valve cataracts on the tube body allow a small amount of air to leak into the dead zone (18) between the cataracts. The air entering the gap cannot pass into the part of lungs with high pressure (16) and goes towards the pharynx (17) through under the valve cataract towards the pharynx. Thanks to it, the air flow into the dead zone established by the cataracts and a more physiological environment is provided. The increase in the amount of bacteria is prevented. A continuous flow towards the pharynx is formed and the movement of liquids coming from in-mouth and stomach into the lungs is prevented. Thanks to it, the risk of infection is decreased.

Through more than two holes (2) on the edge of the tube towards the patient (19), the probability that the holes are obstructed by secretions originating from the lungs is decreased. Moreover, the secretions accumulating in the region between the tubes and the membrane towards the lungs are absorbed inside the endotracheal tube using an aspirator and removed from there.

When the therapy is over and in necessary occasions, the valve cataract balloons are deflated through inflating tube and both valve cataracts are closed so they are closely attached to the walls of endotracheal tube. The tube is easily taken out.

### THE APPLICATION OF THE INVENTION TO THE INDUSTRY

The endotracheal tube without cuffs which serves the purposes mentioned above can be manufactured by companies with a license for constructing medical devices and it is applicable to the industry.

1. The invention is an endotracheal tube without cuffs that has the following components; two valve cataracts which function as cuffs (4,5), the balloons located at the joint of the cataracts and the tube body (6), the framework rods which protect the forms of the valve cataracts (7), the indentation on the tube towards the patient (3), at least three air holes on the edge of the tube towards the patient (2), the ventilation holes on the tube body between two valve cataracts (8), the cleanout holes at the joint of valve cataract towards pharynx and tube body.

2. One of the valve cataracts in the claim 1 is on the edge towards the lungs (4) while the other is on the edge towards the pharynx and have the following characteristics; they prevent air leakage from between trachea and endotracheal tube through unilaterally blocking the air flow, they cover the outside of the tube, they hold the tube body from their top, the bottom edge of them is free, they are parallelly connected to the trachea wall through taking a cylindrical shape, they are made of elastic material, they have membrane structure, their top is in the shape of two cones looking at each other.

3. The framework rods in the claim 1 (7) have the following characteristics; they begin at the joint where valve cataracts (4,%9 are connected to endotracheal tube body (1), they are attached to the cataracts, they become slimmer towards the bottom of the cataract cone, they are made of flexible materials although they are harder than the cataract, they are 6 or 8 pieces which are parallel to each other in equal distances when the cataracts are closed.

4. They are the balloons which open the valve cataracts in the claim 1 and have the following characteristics; they are located at the junction point between the valve cataracts (4,5) and endotracheal tube body (1), they cover the tube body in the shape of ring, they are connected to both tube and the framework rods (7) in the claim 3 within the valve cataracts, there two of them at the junction points where both valve cataracts are connected to the tube, they are connected to valve cataract inflating tubes (9) which enables both inflation and deflation of the balloons.

5. It is the indentation (3) located on the endotracheal tube in the claim 1 towards the patient and has the following characteristics; it is located in the trachea when the valve cataracts (4) in the claim 2 towards the lungs are closed (FIG. 4), it is hidden behind the indentation (3) of the free edge of the valve cataract and it has the level difference which prevents the deformation of the cataract.

6. They are the air holes (2) located on the edge of the tube towards the patient and have the following characteristics; they aspire the lungs originated secretions through inside of the endotracheal tube, they establish gaps which allow the passing of the liquids between the valve cataract towards the lungs (4) and the tube body (1) and they are in the quantity enough to decrease the possibility of blocking the endotracheal tube with lungs-originated secretions.

7. They are ventilation holes (8) located on the tube body between the valve cataracts in the claim 1 and have the following characteristics; they provide the ventilation of the dead-zone (19) between two valve cataracts and trachea walls, they provide the air flow into the dead-zone in the endotracheal tube and from there to the external environment towards the pharynx and they provide 2-4 pieces of small-scale high resistant indentations on the tube body.

8. They are the cleanout holes (10) on the valve cataract towards the pharynx in the claim 1 and have the following characteristics; they are located at the junction where the pharynx side in the claim 2 and the tube body is connected and adjacent to the balloon in the claim 4, they are a few opening facing to valve cataract (5) and endotracheal tube, and their lumen is connected to the cleaning tube (11) and it is ring-shaped.
